(19) United States
(12) Patent Application Publication
(10) Pub. No.: US 2023/0285567 A1
(43) Pub. Date: Sep. 14, 2023

(54) SWELLABLE GELATIN COMPOSITIONS

(71) Applicant: UNIVERSITEIT GENT, Gent (BE)

(72) Inventors: Sandra VAN VLIERBERGHE, Sint-Niklaas (BE); Lana VAN DAMME, Wontergem (BE); Bernard DEPYPERE, Marke (BE); Philip BLONDEEL, Gent (BE)

(21) Appl. No.: 18/002,186

(22) PCT Filed: Jun. 21, 2021
(86) PCT No.: PCT/EP2021/066847

§ 371 Date: Dec. 16, 2022

(30) Foreign Application Priority Data
Jun. 19, 2020 (EP) 20181012

(51) Int. Cl. A61K 47/42

(52) U.S. Cl. CPC A61K 47/42

(57) ABSTRACT

The present invention relates to the field of hydrogels. More specifically, the present invention relates to a swellable composition comprising a gelatin, which gelatin is in a cross-linked state prior to swelling, and wherein said swellable composition is in a multiparticulate form. The present invention further relates to a swelled composition suitable for injection, uses and methods for preparing compositions thereof.

### FIELD OF THE INVENTION

The present invention relates to the field of gelatin hydrogels. More specifically, the present invention relates to swellable compositions comprising crosslinked gelatin, which are in a multiparticulate form and can suitably be used in various applications, such as food industry, cosmetics, human and/or veterinary medicine.

### BACKGROUND TO THE INVENTION

Gelatin is a nature-derived biopolymer material with excellent cell-interactive properties and the potential to form a hydrogel. It has widespread applications in the food and pharmaceutical industry based on its wide availability and cost-efficiency. As a result, the material has become one of the benchmarks in the field of tissue engineering and bio-fabrication. However, since gelatin is characterized by an upper critical solution temperature below the physiological temperature (plus or minus 30° C.), natural gelatin hydrogels are unsuitable for biomedical applications such as tissue engineering. To be suitable in biomedical applications, it is necessary to increase the stability and mechanical properties of gelatin under physiological conditions. Therefore, multiple strategies have emerged to crosslink gelatin, and improve gelatin stability and mechanical properties. The cross-linking methods in the state of the art can be divided in three categories: physical, chemical and enzymatic.

Physical cross-linking methods typically include high energy electron beam, gamma irradiation, plasma treatment and/or dehydrothermal treatment.

Chemical methods include the use of for example EDC/NHS, formaldehyde, glutaraldehyde, genipin and/or (meth)acrylamide. Enzymatic methods include for example microbial transglutaminase methods. Among these methods, chemical cross-linking methods provide for the formation of covalent bonds between gelatin polymeric chains, this results in more stable hydrogels and more controllable mechanical properties. In particular, the use of photo-crosslinking strategies is of specific interest as these methods are generally characterized by relatively mild conditions allowing cell encapsulation in the hydrogel. Additionally, certain (high resolution) additive manufacturing techniques, including stereolithography and two photon polymerization (2PP) require photo-crosslinking to structure the material. The known photo-crosslinking strategies can generally be distinguished into two main categories depending on the crosslinking mechanism: chain-growth polymerization and step-growth polymerization.

Historically, the main part of photo-induced gelatin crosslinking strategies are performed using chain-growth polymerization (radical mediated chain-growth photopolymerization). An often reported gelatin derivative in this respect is gelatin-methacrylamide (Gel-MOD or Gel-MA) in which the primary amine groups of gelatin have been functionalized using methacrylic anhydride yielding crosslinkable methacrylamides. In the last decade, step-growth thiol-ene hydrogels, such as thiol-ene (photo-)click hydrogels have gained increasing interest.

Even though generally cross-linking of functionalized gelatin is advantageous in some applications as it provides for increased in-vivo stability and improved mechanical properties, cross-linked gelatin is not suitable for being injected in that it is not adapted to be injected due to the viscosity of the material obtainable after swelling, which does not allow cross-linked gelatin to be administered through an injection needle.

Currently, some types of functionalized gelatins for tissue engineering applications are injected before being cross-linked and are thereafter cross-linked in-vivo e.g. thiol-ene chemistry modified gelatins. The possibility of cross-linking in-vivo is usually provided by the presence of reactive species, which trigger polymerization and confer cross-linking e.g. photoinitiators. Moreover, stable gelatin-based compositions which are injectable comprise free radicals in-vivo, which cause the presence of reactive oxygen species following implantation. These reactive species can be detrimental for cells.

Other current injectables nowadays use thermo-responsive polymers using trigger materials showing an LCST (Lower Critical Solution Temperature) around body temperature. For these injectables, their synthesis provides for less control over their mechanical properties and their degradation time, which therefore cannot be easily tuned according to needs.

Further injectables in the state of the art include collagen-based fillers. These fillers have several disadvantages. First, collagen-based fillers rapidly degrade in-vivo due to the fact that the physical crosslinks are easily cleaved. Second, cases of allergic reactions after injection have been reported. Gelatin, which is derived from collagen, is less immunogenic than collagen because of the harsh extraction procedure carried out, which results in gelatin providing significantly less adverse allergic effects.

Peptides can be used as an injectable as they exhibit shear thinning effect. However, it is seen that these small chains are quickly degraded in-vivo.

Further injectable fillers in the state of the art, such as hyaluronic acid based fillers e.g. Juvederm® or Restylane® are expensive and do not contain the RGD sequence, also known as arginine, glycine, and aspartate tripeptide, which promotes cell viability due to the interaction with integrins on the cell membrane. Gelatin-based fillers, on the other hand, contain said RGD sequence but are not injectable if not with the disadvantages mentioned hereinabove.

Therefore, even though the advancements in the field have provided for improved functionalized gelatin types and method of synthesis and injection, the state of the art is devoid of gelatin compositions allowing for a less invasive and structurally stable method of delivery, which also overcome the disadvantages mentioned above.

### SUMMARY OF THE INVENTION

It is an object of the present invention to provide for gelatin containing compositions allowing for a less invasive, cost effective and structurally stable method for cell delivery. It is a further object of the present invention to provide modified gelatin avoiding the drawbacks of the prior art. It is a further object of the present invention to provide a hydrogel with controllable swelling and/or water uptake capacity. It is a further object of the present invention to provide for gelatin based compositions that are thermally and chemically stable. Additionally, it is an object of the present invention to provide for gelatin compositions with an improved storage stability in particular at elevated temperatures.

According to a first aspect of the present invention, a swellable composition is provided, wherein the swellable composition comprises a gelatin which is in a cross-linked state prior to swelling, and wherein said swellable composition is in a multiparticulate form, and comprises gelatin particles having a crushed amorphous shape.

According to an embodiment of the present invention, said particles have an average Wadell’s sphericity ϕ up to 0.80. In accordance with a further embodiment of the present invention, said particles have an average Wadell’s sphericity ϕ from 0.20 to 0.80, preferably from about 0.30 to about 0.70, more preferably from about 0.40 to about 0.60.

Further, in accordance with an embodiment of the present invention, said multiparticulate form comprises particles having an average sphericity S from about 1.100. In accordance with a further embodiment of the present invention, said particles have an average sphericity S from about 1.100 to about 1.500, preferably from about 1.200 to about 1.400.

In accordance with a further embodiment of the present invention, said particles have a degree of angularity of at least 140. In accordance with a further embodiment of the present invention, said particles have a degree of angularity from 140 to 950. In accordance with a further embodiment of the present invention, said particles have an average degree of angularity from 250 to 550, preferably from 300 to 500.

In a particular embodiment, the gelatin of the present invention comprises at least a first polymer chain having a cross-linkable functional group in said cross-linked state.

In a further particular embodiment, said multiparticulate form comprises particles having a particle size from about 0.1 µm to 2 mm, preferably 1 µm to 1.5 mm, more preferably 5 µm to about 1 mm, as determined by means of optical microscopy.

In a further particular embodiment, said cross-linkable functional group is selected from: methacrylate, acrylate, methacrylamide, acrylamide, norbornene, maleimide, thiols, EDC, genipin, glutaraldehyde, maleimide, furfuryl, glycidylmethacrylate, preferably methacrylamide, norbornene, thiols and combinations thereof.

In a further particular embodiment, the gelatin further comprises a second polymer chain selected from: synthetic polymers (e.g. PEG), polysaccharides, recombinant chains, proteins, peptides, growth factors and combinations thereof.

In a further particular embodiment, the gelatin comprises a plurality of first and/or second polymer chains.

In accordance with a particular embodiment of the present invention, the gelatin has a degree of substitution from about 10 to 100%.

In accordance with a particular embodiment of the present invention, the gelatin has a cross-linking degree from about 10 to 100%, preferably from 25 to 100%, more preferably from 40 to 100%.

According to a second aspect, the present invention provides for a swelled composition comprising a swellable composition according to present invention and at least one swelling agent.

In a particular embodiment, the swelled composition has a viscosity from about greater than 0 to 200 Pa.s, preferably 10 to 150 Pa.s, more preferably 20 to 120 Pa.s, as determined by means of rheology.

In accordance with a further embodiment of the present invention, the swelled composition has a storage modulus from about 1000 Pa to about 3000 Pa, preferably from about 1500 Pa to about 2500 Pa.

According to a further aspect of the present invention, it is hereby provided a method of preparing a swellable composition, comprising the steps of:
(a) providing a cross-linkable gelatin
(b) cross-linking the gelatin of step (a) to obtain a cross-linked gelatin;
(c) drying the cross-linked gelatin obtained in step (b);
(d) grinding the dried gelatin obtained in step (c) to obtain a multiparticulate form comprising gelatin particles having a crushed amorphous shape;
thereby obtaining the swellable composition as defined in other embodiments according to the present invention.

In an embodiment of the present invention, the method further comprises the step (d) of grinding the dried gelatin thereby obtaining particles having an average Wadell’s sphericity ϕ, from 0.20 to 0.80, preferably from about 0.30 to about 0.70, more preferably from about 0.40 to about 0.60.

In a particular embodiment, the method further comprises the step of: (f) adding to the swelled composition obtained in step (e), a component selected from: (stem) cells, pharmaceutically active compounds or growth factors or combinations thereof.

In a further particular embodiment, in step (b) the gelatin is cross-linked in the form of a film or sheet or crosslinking can be performed in bulk.

In accordance with a further embodiment of the invention, in step (c), the cross-linked gelatin is dried using a method selected from the following non-exclusive list of techniques: air drying, vacuum drying, freeze drying, spray drying. In a preferred embodiment according to the present invention, the cross-linked gelatin is freeze-dried.

In a further particular embodiment, step (d) is performed in the presence of liquid nitrogen.

In a further particular embodiment, in step (d) the cross-linked gelatin is grinded to a particle size from about 0.1 µm to 2 mm, preferably 1 µm to 1.5 mm, more preferably 5 µm to 1 mm.

In a further aspect, the present invention provides for a swellable composition as defined according to other related embodiment, or the swelled composition as defined according to other related embodiments, for use in human and/or veterinary medicine.

In a further aspect, the present invention provides for the use of the swellable composition as defined according to the present invention or the swelled composition as defined according to the present invention; in food industry, cosmetics.

In a particular embodiment, the present invention provides the use of the swellable composition as defined according to the present invention or the swelled composition as defined according to the present invention; in drug delivery and/or cell delivery, and/or as a growth factor delivery.

In a further particular embodiment, the present invention provides for the use of the swellable composition as defined according to the present invention or the swelled composition as defined according to the present invention; as a cosmetic filler.

In a further particular embodiment, the present invention provides for the use of the swellable composition as defined according to the present invention or the swelled composition as defined according to the present invention; in the preparation of a cream or ointment as gelator or thickener.

In a further particular embodiment, the present invention provides for the use of the swellable composition as defined according to the present invention or the swelled composition as defined according to the present invention; as extracellular matrix mimic.

In a further particular embodiment, the present invention provides for the use of the swellable composition as defined according to the present invention or the swelled composition as defined according to the present invention; as composition in tissue engineering applications, such as, and not limited to, aesthetic procedures, large volume tissue reconstruction, small volume tissue reconstruction, fat grafting, lipofilling, burn wounds, dental applications, cartilage and bone tissue engineering, soft tissue engineering, for example adipose, spinal, cardiac,.. tissue engineering, muscle and tendon tissue engineering.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1, also abbreviated as FIG. 1, illustrates mechanical strength measurements of swelled compositions according to the present invention, and how they compare to Juvederm®.

FIG. 2, also abbreviated as FIG. 2, illustrates a graph with cell viability measurements for swelled compositions according to the present invention, and how they compare to Juvederm®.

FIG. 3, also abbreviated as FIG. 3, illustrates two SEM images obtained at a magnification of 495x of swellable gelatin particles in accordance with the present invention.

FIG. 4, also abbreviated as FIG. 4, illustrates optical microscopy images of swellable gelatin particles (left), and swelled gelatin particles (right) in accordance with the present invention.

FIG. 5, also abbreviated as FIG. 5, illustrates optical microscopy images of swellable gelatin particles not in accordance with the present invention, obtained by means of water-in-oil emulsion followed by crosslinking. The obtained particles in FIG. 5 have a characteristic spherical shape. This is in contrast with the shape of particles of the present invention, which is non-spherical.

FIG. 6, also abbreviated as FIG. 6, illustrates the results of viscosity measurements of swollen gelatin particles according to the present invention, at different time points, comparing viscosity at room temperature (RT) and refrigerated (6 - 8° C.).

FIG. 7A, also abbreviated as FIG. 7A, illustrates the results of in vivo testing of the swollen composition combined with cells according to the present invention, wherein FIG. 7B, also abbreviated as FIG. 7B, illustrates the results of in vivo testing of Juvederm® combined with cells.

FIG. 8, also abbreviated as FIG. 8, illustrates the results of storage modulus measurements of swelled gelatin particles according to the present invention, referenced in the legend as particles (light grey), compared to swellable gelatin particles having a spherical shape (as illustrated in FIG. 5), referenced in the legend as spheres (black).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise. The term “about” or “approximately” as used herein when referring to a measurable value such as a parameter, an amount, a temporal duration, and the like, is meant to encompass variations of +/-10% or less, preferably +/-5% or less, more preferably +/- 1% or less, and still more preferably +/-0.1% or less of and from the specified value, insofar such variations are appropriate to perform in the disclosed invention. It is to be understood that the value to which the modifier “about” or “approximately” refers is itself also specifically, and preferably, disclosed. As used in the specification and the appended claims, the singular forms “a”, “an”, and “the” include plural referents unless the context clearly dictates otherwise. By way of example, “a compound” means one compound or more than one compound.

In the context of the present invention, by means of the term “swellable composition”, as used herein, unless indicated otherwise, reference is made to a composition which is capable of increasing its volume by absorbing a swelling agent e.g. a liquid such as water, plasma.

In the context of the present invention, by means of the term “crushed amorphous shape”, when referred to the shape of a particle or a plurality of particles, reference is made to a shape without a clearly defined form, and hence amorphous, which is characteristic of ground or crushed material. Ground or crushed material is characterized by the presence of faces and edges. In the context of the present invention, it is to be understood that the term “crushed amorphous shape” excludes spherical or spheroidal shapes, which cannot be obtained by means of grinding or crushing.

In the context of the present invention, by means of the term “sphericity”, reference is made to a property describing how the shape of a particle, in this case a gelatin particle, compares to the shape of a perfect sphere. The sphericity of a particle can be calculated in various ways, such as the sphericity S and the Wadell’s sphericity ϕ.

In the context of the present invention, by means of the term “sphericity S”, as used herein, reference is made to a property describing how the shape of a particle compares to the shape of a perfect sphere, where a perfect sphere has a sphericity S equal to 1. The sphericity S of a particle is defined as ratio horizontal axis/vertical axis, wherein vertical axis ≤ horizontal axis, wherein the largest particle diameter is defined as the horizontal axis, and the vertical axis is the diameter at a position rotated 90 degrees from the horizontal axis. In accordance with the present invention, the horizontal axis and the vertical axis have been measured on the basis of SEM images of said particles.

In the context of the present invention, by means of the term “Wadell’s sphericity ϕ”, reference is made to a property describing how the shape of a particle compares to the shape of a perfect sphere, where a perfect sphere has a Wadell’s sphericity ϕ equal to 1. The Wadell’s sphericity is defined as:

$$ϕ = \frac{{d}_{c}}{{D}_{c}}$$

wherein d_{c} represents the diameter of the largest inscribing circle, and D_{c} represents the diameter of the smallest circumscribing circle, as described in Wadell, 1935.

In the context of the present invention, by means of the term “angularity”, reference is made to a parameter describing changes in roundness in the corners of a studied particle.

In the context of the present invention, by means of the term “grinding”, reference is made to the reduction of something to small particles or powder by crushing it.

In the context of the present invention, by means of the term “swelled composition”, as used herein, unless indicated otherwise, reference is made to a composition which has increased its volume consequently due to the absorption of a swelling agent e.g. a liquid such as water, plasma, buffer solutions..

In the context of the present invention, by means of the term “gelatin”, as used herein, unless indicated otherwise, reference is made to a biomaterial usually obtained by the hydrolysis of collagen e.g. mammalian and fish collagen or a recombinant gelatin.

In the context of the present invention, by means of the term “cross-linked state”, as used herein, unless indicated otherwise, reference is made to a state characterized by the presence of (covalent) interactions between gelatin polymeric chains, such as via chemical linking. In other words, reference is made to a gelatin which is cross-linked.

In the context of the present invention, by means of the term “multiparticulate form”, as used herein, unless indicated otherwise, reference is made to a form comprising a multitude of particles.

In the context of the present invention, by means of the term “particle size”, as used herein, unless indicated otherwise, reference is made to the average size of the individual particles constituting the multiparticulate form of the compositions according to the present invention, wherein the particle size is calculated such as by means of a sieving process in which we can then specify the grinded particles more accurately. This can also be quantified based on SEM imaging or µCT.

In the context of the present invention, by means of the term “cross-linkable functional group”, as used herein, unless indicated otherwise, reference is made to a functional group situation on a polymer chain capable of providing interaction with another polymer chain. For example, cross-linkable functional groups according to the present invention are, but not limited to, methacrylate, acrylate, methacrylamide, acrylamide, norbornene, maleimide, thiols, EDC, genipin, glutaraldehyde, maleimide, furfuryl, glycidylmethacrylate, preferably methacrylamide, norbornene, thiols and combinations thereof.

In the context of the present invention, by means of the term “water content”, as used herein, unless indicated otherwise, reference is made to the content of water expressed as w/v%, meaning the weight of water relative to the total sample volume, expressed as percentage.

In the context of the present invention, by means of the term “degree of substitution”, as used herein, unless indicated otherwise, reference is made to the amount of cross-linkable moieties/functionalities on a gelatin backbone. This amount can be calculated via a ¹H-NMR spectroscopy, an OPA assay, or ninhydrin assay. The amount of cross-linkable moieties/functionalities on a gelatin backbone can be calculated as described in J. Van Hoorick et al., 2018, A.I. Van Den Bulcke et al., 2000, or S. Van Vlierberghe et al., 2011.

In the context of the present invention, by means of the term “cross-linking degree”, as used herein, unless indicated otherwise, reference is made to the amount of cross-linked moieties on a gelatin backbone, which can be assessed via HRMAS NMR spectroscopy assay, as described in S. Van Vlierberghe et al., 2010.

In the context of the present invention, by means of the term “swelling agent”, as used herein, unless indicated otherwise, reference is made to an agent which is capable of increasing the volume of a swellable composition according to the present invention by absorption of said agent. For example, swelling agents according to the present invention are, but not limited to, water, serum, lipo-aspirate, intravenous fluids, NaCl solution, glucose solution, Hartmann solution, stem cell solution, blood plasma, buffers, such as DMEM, HEPES, and combinations thereof.

The present invention provides for a swellable composition, wherein the swellable composition comprises a gelatin which is in a cross-linked state prior to swelling, and wherein said swellable composition is in a multiparticulate form, and comprises gelatin particles having a crushed amorphous shape, i.e. particles having a non clearly defined shape or form. The gelatin particles in accordance with the present invention have a shape proper to crushed materials such as crushed glass or crushed stone, with edges, faces and/or irregularities. It has been surprisingly found that the present invention provides for a swellable composition having advantageous properties, more specifically of being stable and providing improved injectability after swelling, compared to the state of the art. In particular, swellable particles according to the present invention provide, due to their shape, a more coherent gel (swelled composition). It is believed that swelled particles according to the present invention, obtained from swellable particles of the present invention provide higher external friction forces compared to spherical/spheroidal particles, resulting in a larger resistance against shear deformation and a higher viscosity. Furthermore, the rougher surface and/or the presence of edges will improve cell adhesion and proliferation. This is in contrast with swellable and swelled particles in the state of the art, which have a shape resembling spheres, which does not provide for the aforementioned advantages. According to a preferred embodiment of the present invention, the gelatin particles in the swellable composition according to the present invention have a crushed amorphous shape have an average Wadell’s sphericity ϕ lower than 1, preferably lower than 0.95, preferably lower than 0.90, preferably lower than 0.85, preferably lower than 0.80, preferably lower than 0.75, preferably lower than 0.70, preferably lower than 0.60, preferably lower than 0.55.

According to a preferred embodiment of the present invention, said particles have an average Wadell’s sphericity ϕ up to 0.80. It has been surprisingly found that by providing gelatin particles in accordance with the present embodiment, better injectability after swelling can be provided compared to the state of the art. According to an embodiment of the present invention, said particles have an average Wadell’s sphericity ϕ, from 0.20 to 0.80, preferably from about 0.30 to about 0.70, more preferably from about 0.40 to about 0.60. It has been surprisingly found that by providing gelatin particles in accordance with the present embodiment, better injectability after swelling can be provided.

Further, in accordance with an embodiment of the present invention, said multiparticulate form comprises particles having an average sphericity S from about 1.100, preferably from about 1.100 to about 1.500, preferably from about 1.200 to about 1.400. It has been surprisingly found that by providing gelatin particles in accordance with the present embodiment, better injectability after swelling can be provided.

In accordance with a further embodiment of the present invention, said particles have a degree of angularity of about 140.

In other words, the present invention provides a multiparticulate swellable composition comprising a cross-linked gelatin. Further, the swellable composition according to the present invention may be completely dried, meaning that the final swellable composition has a water content of about 0 w/v%. An advantage of the swellable composition according to the present invention is that it is stable, and when contacted with a swelling agent, the swellable compositions provide for a swelled composition that has a thickness and viscosity allowing for ease of withdrawal from a syringe, in other words, it provides for a swelled composition that can pass through a needle in such a way that a proper amount of it can be more easily injected, in other words, the swelled compositions and swellable composition according to the present invention, provide for an improved injectability. For example, in case of needle diameters from 21 G to 30 G, swellable compositions according to the present invention can be dissolved in either 5-15 w/v% of water to obtain the correct viscosity for ease of injection. However, a person skilled in the art is capable of determining the ideal water content for the compositions, depending on the intended application, and/or envisaged viscosity.

Moreover, swellable compositions according to the present invention are in dried multiparticulate form, and therefore can be easily stored and easily used, due to them requiring simply the contact with a swelling agent, which can be chosen to suit the needs of a specific application in the food industry, cosmetics, human and/or veterinary medicine. Therefore, the swellable compositions according to the present invention are ready-to-use compositions, which do not provide for a labour intense use and/or administration. The swellable compositions according to the present invention provide for improved storage stability in particular at elevated temperatures. In case the swellable compositions are to be injected after swelling, the swellable compositions according to the present invention do not require an in-vivo polymerization step as the compositions are already cross-linked prior to injection. This limits the safety concerns of the compositions, as highly reactive and or radical species are not present in-vivo, but only in a controlled ex-vivo environment. For example, in case UV radiation with UV initiators are used to cross-link gelatin compositions in-vivo, the cross-linked compositions can contain free radicals (oxygen radicals) which are detrimental to the cells. Moreover, the swelled compositions according to the present invention are completely stable at 37° C., allowing for slow and more controlled degradation of the material over time. Further, it has been observed that the crosslinked state is such that some cross-linked compositions in accordance with the present invention are not soluble in water from about 0 to 70° C.

Moreover, the swellable composition according to the present invention comprises gelatin, which acts as a backbone. Gelatin has a low-cost natural backbone, which contains RGD sequences, also known as arginine, glycine, and aspartate tripeptide, which promote cell viability due to the interaction with integrins on the cell membrane.

A further advantage of the swellable composition according to the present invention is that the microparticles will act as a hydrogel by swelling, making it ideal for in-vivo support to get all nutrients to the cells. In accordance with a specific embodiment of the present invention, said multiparticulate form comprises particles having a particle size from about 0.1 µm to 2 mm, preferably 1 µm to 1.5 mm, more preferably 5 µm to about 1 mm. The present particle size has been found advantageous in providing for swelled compositions that can be easily injected. Further, small particle sizes from about 1 µm to 200 µm, provide for faster/easier swelling. In order to provide injectability of the swelled composition through small needles, for example 30 G needles, it is preferred that the particle size of the swellable composition is tuned down to at least 150 µm, preferably at least 100 µm.

The cross-linked state in which the swellable compositions are provided can be obtained by means of a variety of techniques part of the state of the art, such as by physical cross-linking, chemical cross-linking and enzymatic cross-linking. Physical cross-linking methods include energy electron beam, gamma irradiation, plasma treatment and dehydrothermal treatment, whereas chemical cross-linking methods include EDC, formaldehyde, glutaraldehyde, genipin and acrylamide, and others. Enzymatic methods include microbial transglutaminase methods. Among these methods, in particular the use of photo-crosslinking strategies is preferred.

In accordance with a particular embodiment of the present invention, the gelatin has a degree of substitution, which can be calculated/measured for example via the methods described above, from about 10 to 100%.

In accordance with a particular embodiment of the present invention, the gelatin has a cross-linking degree, which can be calculated/measured for example via the methods described above, from about 10 to 100 %, preferably from 25 to 100%, more preferably from 40 to 100%.

In a preferred embodiment of the present invention, the gelatin comprises at least a first polymer chain having a cross-linkable functional group in said cross-linked state. Said at least first polymer chain can provide for increased mechanical stability of the swelled final compositions. The use of cross-linkable functional groups, such as the ones selected from methacrylate, acrylate, methacrylamide, acrylamide, norbornene, maleimide, thiols, EDC, genipin, glutaraldehyde, maleimide, furfuryl, glycidylmethacrylate, preferably methacrylamide, norbornene, thiols and combinations thereof, is capable of providing interaction between polymer chains. The gelatin backbones of the compositions according to the present invention degrade slower over time, and more stable hydrogels and more controllable mechanical properties can be achieved.

In accordance with an embodiment of the present invention, the swelled composition and/or the swellable composition comprise a gelatin comprising cross-linkable functional groups being methacrylamide and norbornene.

Further, in accordance with a preferred embodiment of the present invention, the swelled composition and/or the swellable composition comprise a gelatin comprising cross-linkable functional groups being methacrylamide and norbornene, wherein the gelatin has a degree of substitution of about 66% methacrylamide and 34% norbornene.

Further, in accordance with an embodiment of the present invention, the swelled composition and/or the swellable composition comprise a gelatin comprising cross-linkable functional groups being methacrylamide and norbornene, wherein the gelatin has a degree of substitution of about 39% methacrylamide and 61% norbornene.

In accordance with a further embodiment of the present invention, the gelatin further comprises at least a second polymer chain selected from: synthetic polymers (e.g. PEG), polysaccharides, proteins, peptides, growth factors and combinations thereof. In a further embodiment, the gelatin comprises a plurality of first and/or second polymer chains. The present embodiments allow for further tunability of the mechanical properties of the swellable compositions, that can be designed so to better suit with a specific use. Further, degradability, mechanical strength and swelling properties, can all be improved, or set according to needs, by tuning the material of the compositions by selecting the most appropriate combinations of polymer chain types and functional groups attached to said chains. For example, wherein the particles are required to react in-vivo, the gelatin can be modified e.g. by adding norbornene groups such that the particles can react in-vivo via thiolated compounds present e.g. in plasma. Further, in accordance with the present invention the proposed material of the swellable composition can be provided such that the swelled composition is degradable in-vivo via matrixmetalloproteinases present in the ECM.

According to a further aspect, the present invention also pertains swelled compositions provided by swelling the swellable composition according to any one of the previous embodiments. Accordingly, the present invention provides a swelled composition comprising a swellable composition and a swelling agent. In particular, swelled compositions which are suitable for injection. The swelled compositions according to the present invention provide for similar mechanical strength to Juvederm®. Further, the present swelled compositions can be tuned to achieve even higher mechanical strength if need be, and an improved viability of the encapsulated cells.

In accordance with an embodiment of the present invention, the swelled composition has a viscosity from about greater than 0 to 200 Pa s, preferably 10 to 150 Pa s, more preferably 20 to 120 Pa s.

In accordance with a further embodiment of the present invention, the swelled composition has a storage modulus from about 1000 Pa to about 3000 Pa, preferably from about 1500 Pa to about 2500 Pa.

In accordance with an embodiment of the present invention, the swellable composition can be contacted with a swelling agent so to provide a swelled composition, which swelling agent may be selected from: water, serum, lipo-aspirate, intravenous fluids, NaCl solution, glucose solution, Hartmann solution, stem cell solution, blood plasma, buffers, such as DMEM, HEPES, and combinations thereof. Preferably a lipo-aspirate.

According to a further aspect, the present invention provides a method of preparation of a swellable composition and/or a swelled composition, comprising the steps of: (a) providing a cross-linkable gelatin. For this step, the gelatin can for example be modified so to include a cross-linkable functional group, or provided already modified, in fact, the cross-linkable functional group can be any functional group which is capable of being cross-linked by means of chemical, physical or enzymatic cross-linking methods. Favourably, gelatin is modified to be able to obtain chemically crosslinked films. In order to achieve for better swelling capabilities, in a particular embodiment of the present invention, the gelatin of step (a) has a water content of from about 1 to 40 w/v%, preferably 2 to 30 w/v%, more preferably 5 to 25 w/v%. It has been found that a water content according to the present embodiment provides for better swelling capabilities afterwards. Further, it has been seen that the higher the w/v%, the more difficult it will be to fully dissolve the gelatin in the water, which is of course necessary to be fully dissolved for efficient crosslinking afterwards.

Further step is (b) cross-linking the gelatin of step (a) to obtain a cross-linked gelatin; for this step, the gelatin is cross-linked by means of the most appropriate method to obtain interactions between gelatin chains. Chemical cross-linking methods are preferred. Preferably, both chain and step growth polymerisation methodologies can be used, along with different types of starting materials e.g. having a variety of cross-linkable functional groups and polymer chains. Alternatively, the gelatin can be provided already cross-linked. In case chemically cross-linking methods using UV radiation are used, it has been found favourable to create 2D sheets of the starting material to cross-link, which comprises a photo-initiator, and then applying UV light to the 2D sheet to cross-link the polymeric material, and therefore obtain cross-linked gelatin. In accordance with a further embodiment of the invention, the method further comprises that in step (b) the gelatin provided in step (a) is cross-linked preferably in the form of a film or sheet. Nevertheless, other shapes can also be used. For example, the crosslinking can be performed in bulk, such as wherein the gelatin is e.g. in a flask. The composition of the starting material can be tuned by using different polymerisation techniques or varying the degree of substitution of the gelatin, so that the swellable composition and the swelled composition according to the present invention have specific sought properties.

Further step is (c) drying the cross-linked gelatin obtained in step (b). In accordance with a further embodiment of the invention, in step (c), the cross-linked gelatin is dried using a method selected from the following non-exclusive list of techniques: vacuum drying, air drying, freeze drying, spray drying. In case the cross-linked gelatin is spray dried, a particle formation step would be required. In a preferred embodiment according to the present invention, the cross-linked gelatin is freeze-dried.

Further, the method according to the present invention provides for step (d) grinding the dried gelatin obtained in step (c) to obtain a multiparticulate form having gelatin particles having an average Wadell’s sphericity ϕ up to 0.80, preferably from 0.20 to 0.80, more preferably from about 0.30 to about 0.70, more preferably from about 0.40 to about 0.60; thereby obtaining a swellable composition as defined according to any one of the previous embodiments of the invention.

The present grinding is provided to occur until the desired particle size is achieved. In an advantageous embodiment, liquid nitrogen is poured onto the dried cross-linked gelatin, so to obtain a brittle material, which could then be grinded with ease. The grinding step (d) can take place with or without the presence of liquid nitrogen.

By means of the present method of preparation of a swellable composition and/or a swelled composition, a multiparticulate form characterized by non-spherical particles can be achieved. In contrast with other methods in the state of the art, the method in accordance with the present invention allows the obtainment of cross-linked gelatin particles providing for improved injectability after swelling.

In accordance with an embodiment of the present invention, in step (d) the dried gelatin is grinded to a particle size from about 0.1 µm to 2 mm, preferably 1 µm to 1.5 mm, more preferably 5 µm to 1 mm. This particle size is advantageous, as it allows for ease in administration of the swelled composition. In accordance with a specific embodiment of the present invention, the method further comprises that the step (d) of grinding is performed in the presence of liquid nitrogen.

After the grinding step, a swellable composition according to the present invention is obtained. The swellable composition, which is in multiparticulate form, can be used right away with or without cells or can be stored before use, (e.g. phosphate buffered saline, distilled H₂O, autologous plasma, lipoaspirate, etc.).

In accordance with an embodiment of the invention, it is here provided a method for making a swelled composition, wherein to the method to obtain a swellable composition according to the present invention is further provided a step of (e) adding a swelling agent to said swellable composition obtained in step (d), thereby obtaining a swelled composition. In a particular embodiment, the swelling agent can be selected from the present list comprising, but not limited to: water, serum, lipo-aspirate, intravenous fluids, NaCl solution, glucose solution, Hartmann solution, stem cell solution, blood plasma, buffers, such as DMEM, HEPES, and combinations thereof. Preferably, the swelling agent is a lipo-aspirate.

In accordance with an embodiment of the invention, the method further comprises step (f) of adding to the swelled composition obtained in step (e), a component selected from, but not limited to, stem cells, stromal vascular fractions or combinations thereof. Different components can be added to the swelled compositions e.g. before injection, according to the present invention in order to carry out a specific function. For example, most sensible components, which have to be added separately, can be introduced in the swelled composition after the swelling agent. For example, stromal vascular fractions can be added after the swelling has occurred.

According to a further aspect of the present invention, it is provided the use of the swellable composition as defined in any one of the previous embodiments and/or the swelled composition obtained therefrom, such as, but not limited to, in food industry, cosmetics industry, human and/or veterinary medicine.

In a particular embodiment, in human and/or veterinary medicine the use of the swellable composition and/or the swelled composition therefrom can be for drug delivery and/or cell delivery, but also as a filler, such as a cosmetic filler. In a further particular embodiment of the invention, the swellable composition and/or the swelled composition therefrom, hereby described, can be used in the preparation of a cream or ointment as gelator or thickener e.g. in the food industry as alginates replacement, or in the cosmetic industry in the preparation of a cream or ointment, with no intention of being exhaustive for the possible uses thereon.

In accordance with a preferred embodiment of the present invention, the swellable composition and/or the swelled composition therefrom, are used as extracellular matrix mimic.

In accordance with a further embodiment of the invention, it is hereby provided the use of the swellable composition and/or the swelled composition therefrom as composition in tissue engineering applications, such as, aesthetic procedures, large volume tissue reconstruction, small volume tissue reconstruction, fat grafting, lipofilling, burn wounds, dental applications, cartilage and bone tissue engineering, soft tissue engineering (adipose, spinal, cardiac,..), muscle and tendon tissue engineering.

### EXPERIMENTAL PART

### Material and Methods

Gelatin type B (Gel-B), isolated from bovine skin through an alkaline process was supplied by Rousselot (Ghent, Belgium). 1-Ethyl-3-(3-dimethylaminopropyl)carbodiimide (EDC), N-acetylhomocysteine thiolactone, ethylenediaminetetraacetic acid (EDTA), 5-norbornene-2-carboxylic acid and methacrylic anhydride were purchased from Sigma-Aldrich (Diegem, Belgium). Dimethyl sulfoxide (DMSO) and N-hydroxysuccinimide (NHS) were obtained from Acros (Geel, Belgium). The Spectrapor dialysis membranes MWCO 12.000-14.000 Da were purchased from Polylab (Antwerp, Belgium). Dulbecco’s modified eagle’s medium (DMEM) Glutamax, Fetal bovine serum (FBS), 1% Penicillin/Streptomycin, and TrypLE (consisting of 0.025 Trypsin and 0.01 EDTA) were obtained from Gibco, Life technologies (California, USA). Calcein-acetoxymethyl ester (Calcein-AM), propidium iodide (PI), and Bodipy staining were supplied by Sigma-Aldrich.

Equipment: Freezedryer: Christ freeze-dryer alpha I-5; NMR: A Bruker WH 500 MHz

### Synthesis of GelMODNB

In accordance with the present example, the synthesis of GELMODNB polymers with a low and high degree of substitution is described, in particular, it is described a GELMODNB polymer having DS 39/61 and GELMODNB DS 66/34 (the ratio being methacrylamide/norbornene).

In a first step, gelatin is methacrylated via a protocol first reported by Van den Bulcke et al., 2000.

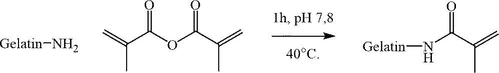

100 g of gelatin B (38.5 mmol amines) was dissolved in 1L phosphate buffer at 40° C. under mechanical stirring. Next, either 1 (5.736 mL; 38.5 mmol) or 0.75 (4.302 mL; 28.875 mmol) equivalents of methacrylic anhydride relative to the primary amines of the gelatin were added after which the solution was stirred vigorously for 1 hour. After 1 hour, 1 L of Milli-Q was added to the reaction mixture. The solution was dialyzed using Milli-RO (spectrapor 4: 12-14 kDa cut off) for 24 hours at 40° C. (water changed 5 times). The solution was then transferred to petri dishes at room temperature to allow the gelatin to gelify. When gelified, the petri dishes were frozen at -20° C. enabling the subsequent removal of ice via lyophilization, obtaining a modified gelatin with a degree of substitution between 60-70%.

Next, the dried GelMOD was used for the next step in which norbornene functionalities will be added on the remaining free amines. To this end, 1.2 equivalents (relative to the primary amines in gelatin type B) of 5-norbornene-2-carboxylic acid were dissolved in 50 ml DMSO at room temperature, see J. Van Hoorick et al., 2018. After complete dissolution, 0.75 equivalents (relative to the amount of amines present in gelatin) of 1-ethyl-3-(3-dimethylaminopropyl)carbodiimide (EDC) were added to the solution followed by degassing. After 5 min, 1.125 equivalents of N-hydroxysuccinimide (NHS) were added to the solution. After 25 h of reaction, the activated solution was added to the GelMOD solution (see below), and allowed to react overnight, so to obtain GelMODNB. To obtain the GelMOD solution, 10 g of GelMOD was dissolved in 150 ml of dry DMSO at 50° C. under inert atmosphere and reflux conditions during 4h. After addition of the activated solution to the GelMOD, the set-up was degassed 3 times, and put under Argon atmosphere. Once the reaction was completed, GelMODNB was obtained. The material was precipitated in a tenfold excess of acetone, filtered on a paper filter of 12-15 µm pore size. Washed with acetone, re-dissolved in milliQ and put in dialysis (spectrapor 4: 12-14 kDa cut off). The water was changed 5 times over the course of 24 hours. After dialysis, the pH of the solution was adjusted to 7.4 using a 1 M NaOH solution resulting in clearing of the solution. Next the material was frozen and lyophilized to remove the water (Christ freeze-dryer alpha I-5). The pressure and temperature inside the freeze-dryer were reduced to 0.37 mbar and -80° C. respectively in order to remove the ice crystals inside the material by sublimation.

GelMODNB having different degrees of substitutions could be obtained by varying the equivalents of methacrylic anhydride and/or 5-norbornene-2-carboxylic acid and others reagents if needed, adapting the procedure hereabove.

### Characterisation of GelMODNB

The primary amine degree of substitution (DS) of gelatin with norbornene and methacrylamide functionalities was quantified via ¹H NMR spectroscopy in accordance with the method described in J. Van Hoorick et al., 2018, and Van Den Bulcke et al., 2000. A Bruker WH 500 MHz was used with deuterium oxide (D₂O) as solvent at 40° C.

### GelMODNB Powder

First, a 10 w/v% solution of the above synthesized material was dissolved at 40° C. in a vial in milliQ. Following complete dissolution, 0.6 mmol of a photo-initiator (lithium phenyl-2,4,6-trimethylbenzoylphosphinate ; Li-TPO) was added. The material was then placed between two glass plates, separated with a 1 mm spacer and put under UV-A (+/- 9.5 mW/cm²) light for 30 min. The total UV-A dose thus includes 17.1 J/cm². Following UV curing, the samples were frozen followed by freeze-drying and grinding of the samples to the desired particle size of 1-150 µm. In the grinding step, liquid nitrogen is used to make the material more brittle.

### Mechanical Analysis

A rheometer (Physica MCR-301; Anton Paar, Sint-Martens-Latem, Belgium) was used to determine the storage modulus (G′), which provides an indication for the elastic properties of the material. A 10 w/v% solution of the powder was made using milliQ water. The material was benchmarked to Juvederm®. 300 µL of the solution was placed between the plates using a gap setting of 0.3 mm. Then, the edges were trimmed and sealed using silicone grease (Mittelviskos) (Bayer, Sigma-Aldrich, Diegem, Belgium) to prevent sample drying. In order to determine the mechanical strength, an oscillation frequency of 1 Hz and a strain of 0.1% were applied as the latter value is within the linear viscoelastic range as determined by isothermal measurements (37° C.) of the storage (G′) and loss moduli (G″) as a function of deformation at a constant frequency (1 Hz) and varying strain (0.01 - 10%).

### Cell Encapsulation Experiments

Human Adipose Stromal Cells, or ASCs, isolated from lipoaspirate were used for the cell encapsulation experiments. To this end, the material was swollen in sterile PBS at a 10 w/v%. Cell encapsulation was performed by mixing the swellable composition with ASC’s at a density of 10.000 cells/100 µL. A 96-well plate was used to inject 100 µL of the material to which an additional 100 µL of medium was added. The material was benchmarked to Juvederm® and Tissue culture plastic (TCP) in which 10.000 cells were incorporated in 100 µL of the material or seeded directly onto a well plate respectively. The well plate was placed in an incubator at 37° C. and in the presence of 5% CO₂.

For the differentiation assays, the basic culture medium (DMEM, 10% FBS, 1% penicillin/streptomycin) was replaced with adipogenic differentiation medium (DMEM, 10% FBS, 1% penicillin/streptomycin, 1 IM dexamethasone, 200 IM indomethacin, 10 Ig/mL insulin, 0.5 mM IBMX) after 48 h. The medium was refreshed every 2-3 days.

### Live/dead Cell Viability Assay

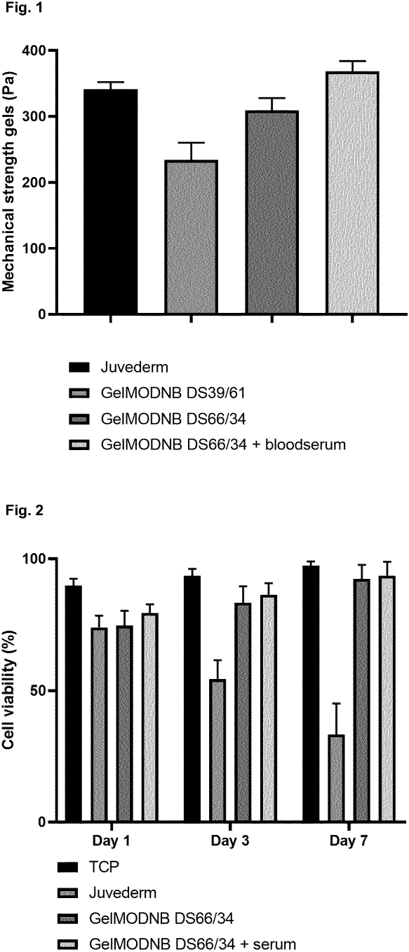

The cytocompatibility of incapsulated materials obtained above was tested through a live/dead viability assay using Calcein acetoxymethyl ester (Calcein-AM) and propidium iodide (PI) staining. For every 1 mL PBS, 2 µl Calcein-AM and 2 µl PI were added. A 96-well plate was used and 0.15 mL of the solution was added to each well. The wells were incubated in the dark by placing them under aluminium foil for 10 minutes at room temperature. A fluorescence microscope with a green fluorescent protein (GFP) filter for Calcein was used to visualize the living cells. A Texas Red (TxRed) filter was applied to visualize the dead cells using PI. Quantification of the live/dead ratio was realized via ImageJ software, enabling a cell count of both living and dead cells. The related obtained graph results of the cell viability assays are shown in FIG. 2.

### Adipogenic Differentiation

First, a stock solution of the Bodipy (493/503) dye of 0.5 mg/mL (1.9 mM) was made in ethanol (100%). When Bodipy was fully dissolved, the stock solution was stored in Eppendorf tubes, frozen and protected from light. Fluorescence staining was done by thawing an Eppendorf vial containing the stock solution and dissolving 12 mL in 1 mL serum-free medium. The solution was mechanically emulsified by vigorously mixing. Bodipy staining of the cells was realized by adding 75 µL mL Bodipy solution to 75 µL fresh medium in the 96 well plate containing the material and incubated for 15 min. Imaging was performed through a fluorescence microscope with a GFP filter.

### SEM Imaging

The morphology of the gelatin particles was obtained using a Phenom FEI (tungsten hairpin electron gun, backscattered electron detector) scanning electron microscope. To avoid charging of the samples, a gold coating plasma magnetron sputter coating) was applied on the surface of the samples prior to the measurements (Automatic Sputter Coater K550X with a RV3 two stage rotary vane pump).

### Sphericity and Angularity

Calculations of sphericity were performed using ImageJ software of the obtained SEM images. To this end, the sphericity S was calculated by measuring the largest particle diameter and the particle diameter at a position rotated 90 degrees. The sphericity S of a particle is defined as ratio horizontal axis/vertical axis, wherein vertical axis ≤ horizontal axis.

The Wadell’s sphericity ϕ was measured by drawing the largest inscribing circle and smallest circumscribing circle in the software and measuring the diameter of these obtained circles. The Wadell’s sphericity is defined as:

$$ϕ = \frac{{d}_{c}}{{D}_{c}}$$

Where d_{c} represents the diameter of the largest inscribing circle, and D_{c} represents the diameter of the smallest circumscribing circle. The angularity of the particles was obtained through a method first described by Lees et al., 1964, as defined by:

$${A}_{i} = \left(180 ° - \text{α}\right) \frac{x}{r}$$

Wherein αrepresents the measured angle, x the distance of the tip of the corner from the center of the maximum inscribed circle and r the radius of the maximum inscribed circle. All measurements of Wadell’s sphericity Φ, Sphericity S and angularity were performed on 25 particles.

### Stability Data

A rheometer (Physica MCR-301; Anton Paar, Sint-Martens-Latem, Belgium) was used to determine the storage modulus (G′), and the viscosity at predefined time points. A 10 w/v% solution of the powder was made using milliQ water. 300 µL of the solution was placed between the plates using a gap setting of 0.3 mm. In order to determine the mechanical strength, an oscillation frequency of 1 Hz and a strain of 0.1% were applied. The viscosity was measured as a function of shear rate, which varied between 0.1 - 1000 s⁻¹. Stability measurements were performed in triplicate.

### In Vivo Data

Swiss nude mice were used to perform a submammary injection of the swollen composition in the presence of the stromal vascular fraction and adipocytes derived from human lipo-aspirate, compared to Juvederm® combined with the cells. After 3 months, the mice were sacrificed and ex vivo histology was performed. All samples were fixed overnight in 4% paraformaldehyde, dehydrated in graded alcohol series, cleared with toluene and embedded in paraffin. Using a microtome (Reichert-Jung 2040) samples were sectioned at 5 µm thickness and subsequently deparaffinized, rehydrated and stained. For evaluation of overall morphology, a Haematoxylin/Eosin (HE) (VWR, ThermoFisher was carried out according to standard protocols.

### Comparisons Data - Spherical vs Present Invention

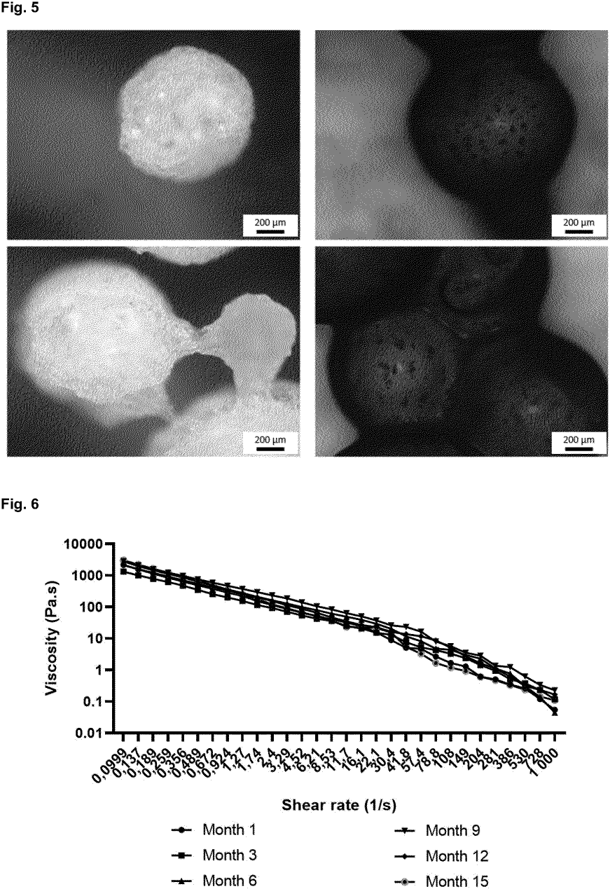

Further, FIG. 8 illustrates comparative results of storage modulus measurements of swelled gelatin particles according to the present invention, referenced in the legend as particles (light grey), compared to swellable gelatin particles having a spherical shape obtained by means of water-in-oil emulsion (as illustrated in FIG. 5), A rheometer (Physica MCR-301; Anton Paar, Sint-Martens-Latem, Belgium) was used to determine the storage modulus (G′), which provides an indication for the elastic properties of the material. A 10 w/v% solution of the powder was made using milliQ water. The material was benchmarked to spherical particles obtained via water in oil emulsion of the same starting material as the particles described in the present invention. To determine the mechanical strength, an oscillation frequency of 1 Hz and a strain of 0.1% were applied.

### Results

### Mechanical Analysis Results

The obtained gels were compared to Juvederm® with respect to mechanical properties and here we could see that the GelMODNB gels obtained, meaning GelMODNB DS39/61 (the gelatin having degree of substitution of 39% methacrylamide and 61% norbornene), GelMODNB DS66/34 (the gelatin having degree of substitution of 66% methacrylamide and 34% norbornene) and GelMODNB DS66/34 with serum, have a similar mechanical strength compared to the benchmark Juvederm®, with the GelMODNB DS66/34 variant being the best performing. See FIG. 1.

FIG. 1 shows that the mechanical strength of GelMODNB gels is comparable to that of Juvederm ® which is already well defined as a commercially available cosmetic filler. Depending on the liquid in which the material swells and the crosslinkable moieties, we can see that there are differences present in the mechanical properties (e.g. between the DS39/61 variant and the DS66/34 variant). These differences suggest that the number of crosslinkable moieties is of importance to obtain a good injectable filler system.

### Cell Viability Assay Results and Adipogenic Differentiation

The cell viability of encapsulated adipose derived stem cells was assessed for TCP, Juvederm®, GelMODNB DS66/34, and GelMODNB DS66/34 with serum, based on a live/dead staining with calcein-AM and Propidiumiodide on different time points (at 1 day, 3 days, 7 days). Here we observed good cell morphology on our materials with cell proliferation and a superior viability compared to the benchmark Juvederm®. See FIG. 2 for the graphs. FIG. 2 shows in the graph a significant increase in cell viability compared to Juvederm®. The cell viability % illustrates a clear superiority of the material according to the present invention at days 3 and in particular day 7.

Imaging pertaining to cell viability (not shown) related to the assay provided in FIG. 2, further illustrate that GelMODNB DS66/34, and GelMODNB DS66/34 with serum provide for better cell viability, in particular, an increase in the number of cells present in the present material compared to Juvederm is visible. The better cell viability allows the materials according to the present invention to be used advantageously e.g. for tissue engineering purposes. The imaging further shows that the material will act as an ECM mimic during the time that cells can begin to develop their own ECM.

Further imaging pertaining to the adipogenic differentiation (not shown), illustrates that encapsulated cells in the materials according to the present invention spread into their correct morphology and possess good proliferation potential, which can be attributed to both biocompatibility of the material as well as the presence of RGD sequences. Further, differentiation imaging shows the differentiation of the adipose derived stem cells into the adipogenic lineage. Bodipy staining was used to assess the intracellular lipid droplets only present in (pre-)adipocytes. Here, it could be observed that there were more differentiated cells present in the GelMODNB DS 66/34 compared to Juvederm, most likely related to the low viability at day 7 of the cells encapsulated in Juvederm.

### SEM Imaging and Sphericity

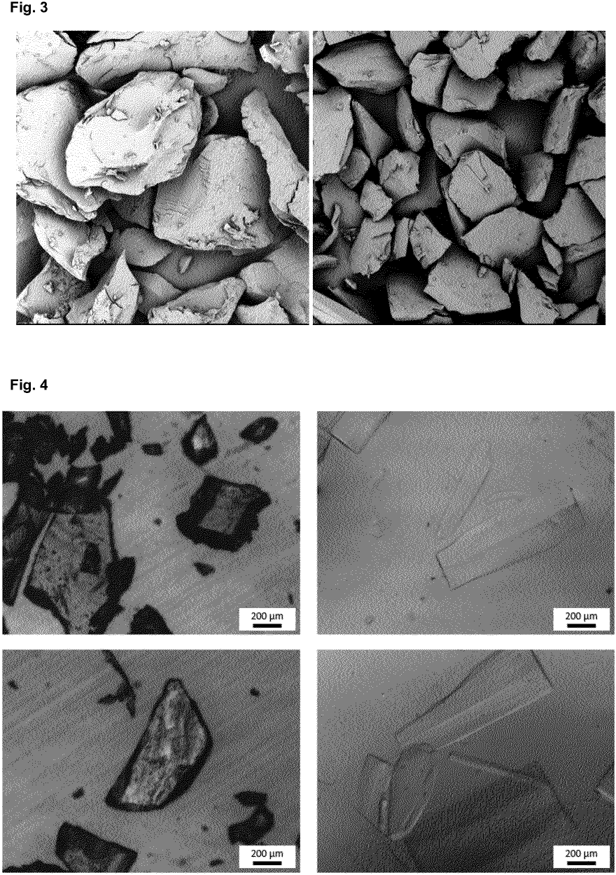

As can be seen on the SEM imaging, FIG. 3, irregularly shaped particles with an edged surface and a high degree of angularity were obtained. To this end, Wadell’s sphericity was calculated and an average value of 0.46 ± 0.28 was obtained for particles in accordance with the present invention. Moreover, their shape shape was further measured to having an average sphericity S of 1.302 ± 0.2159. Lastly, the average angularity Aᵢ of the samples was calculated to be 406.6 ± 271.9. Further, FIG. 4, illustrates optical microscopy images of swellable gelatin particles (left), and swelled gelatin particles (right) in accordance with the present invention.

Further, FIG. 5, illustrates optical microscopy images of swellable gelatin particles not in accordance with the present invention, obtained by means of water-in-oil emulsion followed by crosslinking. The Wadell’s sphericity of these spherical particles obtained by means of water-in-oil emulsion was also measured, and an average Wadell’s sphericity value of 0.93 ± 0.04 was obtained. The difference in Wadell’s sphericity clearly shows that gelatin particles obtained via water-in-oil emulsion have a shape that resembles the one of a sphere, in contrast with gelatin particles in accordance with the present invention.

### Stability

The dried particles have been stored for multiple months in a dry state either at room temperature or in the fridge (6 - 8° C.). The obtained values of storage modulus can be observed in the table here below:

Mechanical properties (Pa)

| | Fridge | Room temperature |
|---|---|---|
| Month 1 | 3430 ± 568 | 3120 ± 369 |
| Month 3 | 3240 ± 247 | 2846 ± 485 |
| Month 6 | 3330 ± 342 | 3159 ± 147 |
| Month 9 | 2930 ± 232 | 2597 ± 587 |
| Month 12 | 3156 ± 477 | 2987 ± 236 |
| Month 15 | 3867 ± 355 | 3548 ± 422 |

No significant differences could be observed nor over time nor between both preservation methods. It could thus be concluded that the swellable composition is stable over time. The measurements of viscosity for the swelled compositions is illustrated in FIG. 6.

### In Vivo Data

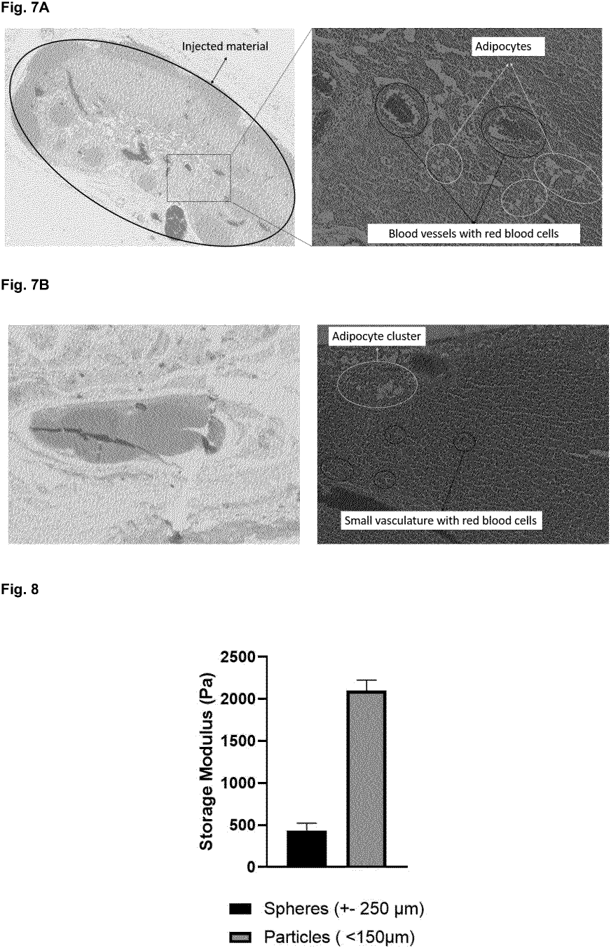

Due to the higher resistance to shear deformation, the swollen composition remained localized, as can be seen in FIG. 7A. Furthermore, compared to the benchmark Juvéderm (FIG. 7B), good vascularisation can be observed as well as multiple adipocyte clusters.

### Comparisons Data - Spherical Shape vs Present Invention

Further, FIG. 8 illustrates comparative results of storage modulus measurements of swelled gelatin particles according to the present invention, referenced in the legend as particles (light grey), compared to swellable gelatin particles having a spherical shape obtained by means of water-in-oil emulsion (as illustrated in FIG. 5), referenced in the legend as spheres (black). The present results show the positive effect the crushed amorphous shape of the gelatin particles provides to a swelled composition, in comparison with a swelled composition comprising spherical particles. The swelled composition from the present invention, obtained a higher mechanical strength compared to these of the spherical particles. As both base material, water content and crosslinking degree are similar, the increase in storage modulus can be explained by a higher external friction force, resulting in a more coherent gel. These high friction forces will be directly correlated to the larger resistance against shear deformation.

### REFERENCES

1. J. Van Hoorick, P. Gruber, M. Markovic, M. Rollot, G.J. Graulus, M. Vagenende, M. Tromayer, J. Van Erps, H. Thienpont, J.C. Martins, S. Baudis, A. Ovsianikov, P. Dubruel, S. Van Vlierberghe, Highly reactive thiol-norbornene photo-click hydrogels: toward improved processability, Macromol. Rapid Commun. 39 (2018) 1-7, https://doi.org/10.1002/marc.201800181

2. A.I. Van Den Bulcke, B. Bogdanov, N. De Rooze, E.H. Schacht, M. Cornelissen, H. Berghmans, Structural and rheological properties of methacrylamide modified gelatin hydrogels, Biomacromolecules 1 (2000) 31-38, https://doi.org/ 10.1021/bm990017d.

3. S. Van Vlierberghe, E. Schacht, P. Dubruel, Reversible gelatin-based hydrogels: Finetuning of material properties, Eur. Polym. J. 47 (2011) 1039-1047, https:// doi.org/10.1016/j.eurpolymj.2011.02.015.

4. Van Vlierberghe, S., Fritzinger, B., Martins, J. C., & Dubruel, P. (2010). Hydrogel Network Formation Revised: High-Resolution Magic Angle Spinning Nuclear Magnetic Resonance as a Powerful Tool for Measuring Absolute Hydrogel Cross-Link Efficiencies. Applied Spectroscopy, 64(10), 1176-1180. https://doi.org/10.1366/000370210792973550.

5. Wadell, H. (1935). Volume, Shape, and Roundness of Quartz Particles. The Journal of Geology, 43(3), 250-280. doi:10.1086/624298.

6. LEES, G. (1964), A new method for determining the angularity of particles. Sedimentology, 3: 2-21. https://doi.org/10.1111/j.1365-3091.1964.tb00271.x

1. A swellable composition, comprising:
a gelatin which is in a cross-linked state prior to swelling, and
wherein said swellable composition is in a multiparticulate form, and comprises gelatin particles having a crushed amorphous shape.

2. The swellable composition according to claim 1, wherein said particles have an average Wadell’s sphericity ϕ up to 0.80.

3. The swellable composition according to claim 2, wherein said particles have an average Wadell’s sphericity ϕ, from 0.20 to 0.80, preferably from about 0.30 to about 0.70, more preferably from about 0.40 to about 0.60.

4. The swellable composition according to any one of claims 1 to 3, wherein the gelatin comprises at least a first polymer chain having a cross-linkable functional group in said cross-linked state.

5. The swellable composition according to claim 1 to 4, wherein said multiparticulate form comprises particles having a particle size from about 0.1 µm to 2 mm, preferably 1 µm to 1.5 mm, more preferably 5 µm to about 1 mm, as determined by means of optical microscopy.

6. A swelled composition comprising the swellable composition according to any one of claims 1 to 5 and at least one swelling agent.

7. The swelled composition according to claim 6, wherein the swelled composition has a viscosity from about greater than 0 to 200 Pa s, preferably 5 to 150 Pa s, more preferably 10 to 120 Pa s, as determined by means of rheology.

8. A method of preparing a swellable composition as defined in anyone of claims 1 to 5, comprising the steps of:
(a) providing a cross-linkable gelatin
(b) cross-linking the gelatin of step (a) to obtain a cross-linked gelatin;
(c) drying the cross-linked gelatin obtained in step (b);
(d) grinding the dried gelatin obtained in step (c) to obtain a multiparticulate form comprising gelatin particles having a crushed amorphous shape;
thereby obtaining the swellable composition as defined in anyone of claims 1 to 5.

9. The method of preparing a swellable composition according to claim 8, wherein step (d) further comprises grinding the dried gelatin thereby obtaining particles having an average Wadell’s sphericity ϕ up to 0.80, preferably from 0.20 to 0.80, more preferably from about 0.30 to about 0.70, more preferably from about 0.40 to about 0.60.

10. A method of preparing a swelled composition as defined in any one of claims 8 to 9, comprising applying the method according to any one of claims 8 to 9 and:
(e) adding a swelling agent to said swellable composition obtained in step (d), thereby obtaining a swelled composition.

11. The method according to claim 10, further comprising the step of:
(f) adding to the swelled composition obtained in step (e), a component selected from: cells, stem cells, pharmaceutically active compounds, growth factors, or combinations thereof.

12. The method according to any one of claims 8 to 11, wherein in step (b) the gelatin is cross-linked in the form of a film or sheet.

13. The method according to any one of claims 8 to 12, wherein in step (c), the cross-linked gelatin is freeze dried.

14. The swellable composition as defined in any one of claims 1 to 5, or the swelled composition as defined in any one of claims 6 to 7, for use in human and/or veterinary medicine.

15. Use of the swellable composition as defined in any one of claims 1 to 5, or the swelled composition as defined in any one of claims 6 to 7; in food industry, cosmetics, drug delivery and/or cell delivery and/or as a growth factor delivery, and/or as a cosmetic filler, in the preparation of a cream or ointment as gelator or thickener, as extracellular matrix mimic, as composition in tissue engineering applications, such as, aesthetic procedures, large volume tissue reconstruction, small volume tissue reconstruction, fat grafting, lipofilling, burn wounds, dental applications, cartilage and bone tissue engineering, soft tissue engineering, such as adipose, spinal, cardiac tissue engineering, muscle and tendon tissue engineering.
